(19) United States
(12) Patent Application Publication
(10) Pub. No.: US 2008/0234625 A1
(43) Pub. Date: Sep. 25, 2008

(54) Fuse Assembly For Single Use Medical Device

(76) Inventors: Bruno Dacquay, Irvine, CA (US); Cesario Dos Santos, Aliso Viejo, CA (US); Casey Lind, Irvine, CA (US)

Correspondence Address: ALCON, IP LEGAL, TB4-8, 6201 SOUTH FREEWAY, FORT WORTH, TX, 76134, US

(21) Appl. No.: 11/688,489

(22) Filed: Mar. 20, 2007

(51) Int. Cl. A61N 1/30

(52) U.S. Cl. 604/19

(57) ABSTRACT

A disposable injection device includes a dispensing chamber, a plunger, a fuse in series with a light, and a housing. The dispensing chamber has an inner surface and an outer surface. The inner surface defines a cavity for receiving a quantity of a substance. The plunger is engaged with the inner surface of the dispensing chamber, is capable of sliding in the cavity of the dispensing chamber, and is fluidly sealed to the inner surface of the dispensing chamber. The housing at least partially encloses the dispensing chamber and the plunger. After the substance has been delivered from the dispensing chamber, the fuse is blown causing the light to go out and disabling the device.

### RELATED APPLICATIONS

This Application is a continuation-in-part of U.S. patent application Ser. No. ______, which claims priority to U.S. Provisional Patent Application No. ______, converted from U.S. patent application Ser. No. 11/581629 filed Oct. 16, 2006, and which is a continuation-in-part of U.S. patent application Ser. No. 11/435,906 filed May 17, 2006.

### BACKGROUND OF THE INVENTION

The present invention relates to a single-use medical device and more particularly to a two-piece ophthalmic drug delivery device with a disposable tip end containing a fuse assembly.

Several diseases and conditions of the posterior segment of the eye threaten vision. Age related macular degeneration (ARMD), choroidal neovascularization (CNV), retinopathies (e.g., diabetic retinopathy, vitreoretinopathy), retinitis (e.g., cytomegalovirus (CMV) retinitis), uveitis, macular edema, glaucoma, and neuropathies are several examples.

These, and other diseases, can be treated by injecting a drug into the eye. Such injections are typically manually made using a conventional syringe and needle. FIG. 1 is a perspective view of a prior art syringe used to inject drugs into the eye. In FIG. 1, the syringe includes a needle 105, a luer hub 110, a chamber 115, a plunger 120, a plunger shaft 125, and a thumb rest 130. As is commonly known, the drug to be injected is located in chamber 115. Pushing on the thumb rest 130 causes the plunger 120 to expel the drug through needle 105.

In using such a syringe, the surgeon is required to puncture the eye tissue with the needle, hold the syringe steady, and actuate the syringe plunger (with or without the help of a nurse) to inject the fluid into the eye. The volume injected is typically not controlled in an accurate manner because the vernier on the syringe is not precise relative to the small injection volume. Fluid flow rates are uncontrolled. Reading the vernier is also subject to parallax error. Tissue damage may occur due to an “unsteady” injection.

An effort has been made to control the delivery of small amounts of liquids. A commercially available fluid dispenser is the ULTRA™ positive displacement dispenser available from EFD Inc. of Providence, R.I. The ULTRA dispenser is typically used in the dispensing of small volumes of industrial adhesives. It utilizes a conventional syringe and a custom dispensing tip. The syringe plunger is actuated using an electrical stepper motor and an actuating fluid. With this type of dispenser, the volumes delivered are highly dependent on fluid viscosity, surface tension, and the specific dispensing tip. Parker Hannifin Corporation of Cleveland, Ohio distributes a small volume liquid dispenser for drug discovery applications made by Aurora Instruments LLC of San Diego, Calif. The Parker/Aurora dispenser utilizes a piezo-electric dispensing mechanism. While precise, this dispenser is expensive and requires an electrical signal to be delivered to the dispensing mechanism.

U.S. Pat. No. 6,290,690 discloses an ophthalmic system for injecting a viscous fluid (e.g. silicone oil) into the eye while simultaneously aspirating a second viscous fluid (e.g. perflourocarbon liquid) from the eye in a fluid/fluid exchange during surgery to repair a retinal detachment or tear. The system includes a conventional syringe with a plunger. One end of the syringe is fluidly coupled to a source of pneumatic pressure that provides a constant pneumatic pressure to actuate the plunger. The other end of the syringe is fluidly coupled to an infusion cannula via tubing to deliver the viscous fluid to be injected.

It would be desirable to have a portable hand piece for injecting a drug into the eye. Such a hand piece has a limited reuse assembly attachable to and removable from a disposable tip segment. The disposable tip segment contains the drug, a needle for administering the drug, and various other components. In order to prevent infection, the needle of tip segment needs to be sterile. The tip segment could come prepackaged in a sterile pack. In order to insure that the disposable tip segment is used only once, it would be desirable to have a fused light that prevents the reuse of the disposable tip segment.

### SUMMARY OF THE INVENTION

In one embodiment consistent with the principles of the present invention, the present invention is a disposable injection device including a dispensing chamber, a plunger, a fuse in series with a light, and a housing. The dispensing chamber has an inner surface and an outer surface. The inner surface defines a cavity for receiving a quantity of a substance. The plunger is engaged with the inner surface of the dispensing chamber, is capable of sliding in the cavity of the dispensing chamber, and is fluidly sealed to the inner surface of the dispensing chamber. The housing at least partially encloses the dispensing chamber and the plunger. After the substance has been delivered from the dispensing chamber, the fuse is blown causing the light to go out and disabling the device.

In another embodiment consistent with the principles of the present invention, the present invention is an ophthalmic injection system including a tip segment and a limited reuse assembly. The tip segment includes a dispensing chamber, a plunger, a fuse in series with a light, and a housing. The dispensing chamber has an inner surface and an outer surface. The inner surface defines a cavity for receiving a quantity of a substance. The plunger is engaged with the inner surface of the dispensing chamber, is capable of sliding in the cavity of the dispensing chamber, and is fluidly sealed to the inner surface of the dispensing chamber. The plunger also has an end with a first mechanical linkage interface. The housing at least partially encloses the dispensing chamber and the plunger. The limited reuse assembly includes a power source, a controller for controlling the operation of the system, a actuator having a shaft, a second mechanical linkage interface located on an end of the shaft, and a second housing at least partially enclosing the controller and the actuator. After the substance has been delivered from the dispensing chamber, the fuse is blown causing the light to go out and disabling the device.

In another embodiment consistent with the principles of the present invention, the present invention is a method of operating an ophthalmic injection system. The method includes recognizing a connection between a tip segment and a limited reuse assembly and determining if a fuse has been blown. If the fuse has not been blown, then a light is illuminated indicating that the tip segment is ready to be used. After the tip segment has been used, a current to the fuse is increased thus causing the fuse to be blown.

In another embodiment consistent with the principles of the present invention, the present invention is a medical device having a single use component and a limited reuse assembly. The single-use component has a fuse connected in series with a light and an interface for receiving electric current that is provided to the fuse and the light. The limited reuse assembly is attachable to and removable from the single-use component. The limited reuse assembly has a controller for controlling the operation of the single-use component and a power source for providing the electric current to the fuse and the light via the interface. After the single-use component has been used, the fuse is blown thus disabling the single use component.

In another embodiment consistent with the principles of the present invention, the present invention is a single-use medical device including a fuse connected in series with a light and an interface for receiving electric current. The electric current is provided to the fuse and the light. After the device has been used, the fuse is blown thus disabling the device.

It is to be understood that both the foregoing general description and the following detailed description are exemplary and explanatory only and are intended to provide further explanation of the invention as claimed. The following description, as well as the practice of the invention, set forth and suggest additional advantages and purposes of the invention.

### BRIEF DESCRIPTION OF THE DRAWINGS

The accompanying drawings, which are incorporated in and constitute a part of this specification, illustrate several embodiments of the invention and together with the description, serve to explain the principles of the invention.

FIG. 1 is a perspective view of a prior art syringe.

FIG. 2 is a view of an ophthalmic hand piece including a drug delivery tip segment and a limited reuse assembly according to an embodiment of the present invention.

FIG. 3 is a diagram of a fused light for use in a drug delivery tip segment according to an embodiment of the present invention.

FIG. 4 is an exploded cross section view of a drug delivery tip segment for an ophthalmic hand piece according to an embodiment of the present invention.

FIG. 5 is cross section view of a drug delivery tip segment and a limited reuse assembly according to an embodiment of the present invention.

FIG. 6 is a flow chart depicting one method of operation of the present invention.

### DETAILED DESCRIPTION OF THE PREFERRED EMBODIMENTS

Reference is now made in detail to the exemplary embodiments of the invention, examples of which are illustrated in the accompanying drawings. Wherever possible, the same reference numbers are used throughout the drawings to refer to the same or like parts.

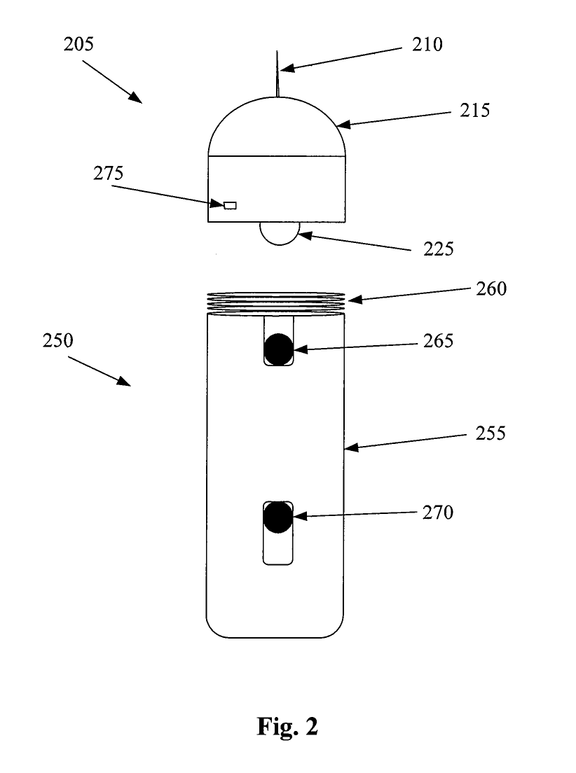

FIG. 2 depicts one view of an ophthalmic hand piece including a drug delivery tip segment and a limited reuse assembly according to an embodiment of the present invention. In FIG. 2, the hand piece includes a tip segment 205 and a limited reuse assembly 250. The tip segment 205 includes a needle 210, a housing 215, a plunger connection 225, and an optional light 275. The limited reuse assembly 250 includes a housing 255, a switch 270, a lock mechanism 265, and a threaded portion 260.

Tip segment 205 is capable of being connected to and removed from Limited reuse assembly 250. In this embodiment, tip segment 205 has a threaded portion on an interior surface of housing 215 that screws onto the threaded portion 260 of limited reuse assembly 250. In addition, lock mechanism 265 secures tip segment 215 to limited reuse assembly 250. Lock mechanism 265 may be in the form of a button, a sliding switch, or a cantilevered mechanism. Other mechanisms for connecting tip segment 205 to limited reuse assembly 250, such as those involving structural features that mate with each other, are commonly known in the art and are within the scope of the present invention.

Needle 210 is adapted to deliver a substance, such as a drug, into an eye. Needle 210 may be of any commonly known configuration. Preferably, needle 210 is designed such that its thermal characteristics are conducive to the particular drug delivery application. For example, when a heated drug is to be delivered, needle 210 may be relatively short (several millimeters) in length to facilitate proper delivery of the drug.

Switch 270 is adapted to provide an input to the system. For example, switch 270 may be used to activate the system or to turn on a heater. Other switches, buttons, or user-directed control inputs are commonly known and may be employed with limited reuse assembly 250 and/or tip segment 205.

Optional light 275 is illuminated when tip segment 205 is ready to be used. Optional light 275 may protrude from housing 215, or it may be contained within housing 215, in which case, optional light 275 may be seen through a clear portion of housing 215. In other embodiments, optional light 275 may be replaced by an indicator, such as a liquid crystal display, segmented display, or other device that indicates a status or condition of the tip segment. For example, optional light 275 may also pulse on and off to indicate other states such as but not limited to a system error, fully charged battery, insufficiently charged battery or faulty connection between the tip segment 205 and limited use assembly 250.

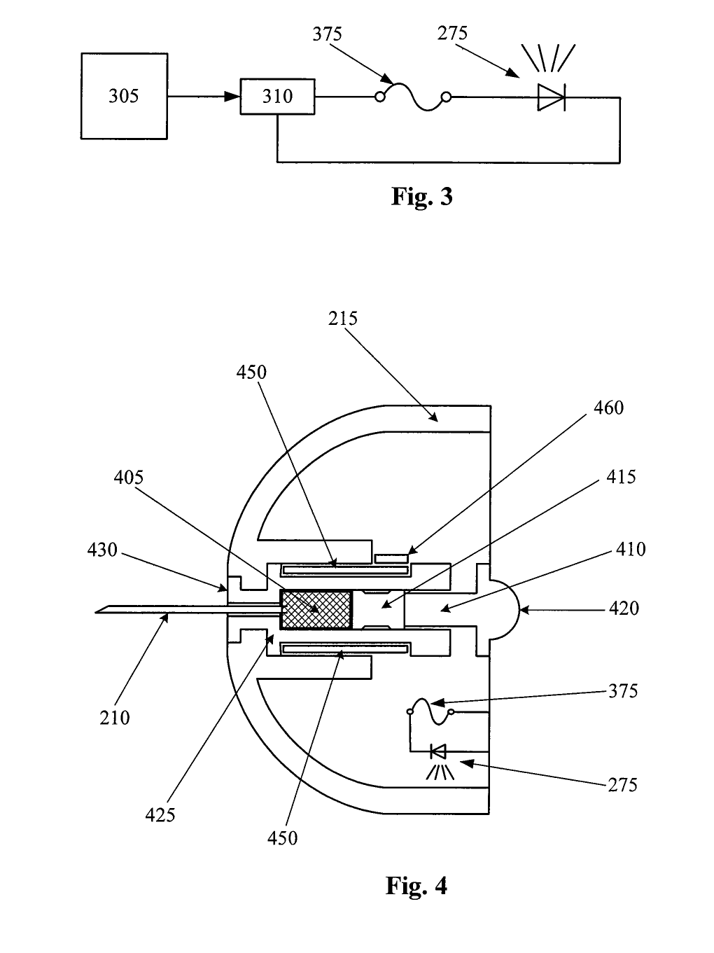

FIG. 3 is a diagram of a fused light for use in a drug delivery tip segment according to an embodiment of the present invention. In FIG. 3, optional light 275 and fuse 375 are connected in series with power source 310. Controller 305 controls the operation of power source 310.

In the embodiment of FIG. 3, optional light 275 is a light emitting diode of any appropriate color. In other embodiments, optional light 275 may be a lamp, a phosphorescent light, or any other similar electric or electronic light source. In other embodiments, optional light 275 is any type of indicator, such as a liquid crystal display or a segmented display.

Fuse 375 is a fuse with a current rating greater than the operating current of optional light 275. Fuse 375 may be a common glass encapsulated fuse, a trace fuse on a printed circuit board, or other similar structure that provides the function of a fuse. For example, a switch or switching circuit may be used to provide the functionality of fuse 375.

Power source 310 is typically a rechargeable battery with associated electronics. In other cases, power source 310 is a disposable battery or simply a connection to an independent power source, such as a switch mode power supply. In this embodiment, power source 310 also includes the charging and current driving electronics associated with it.

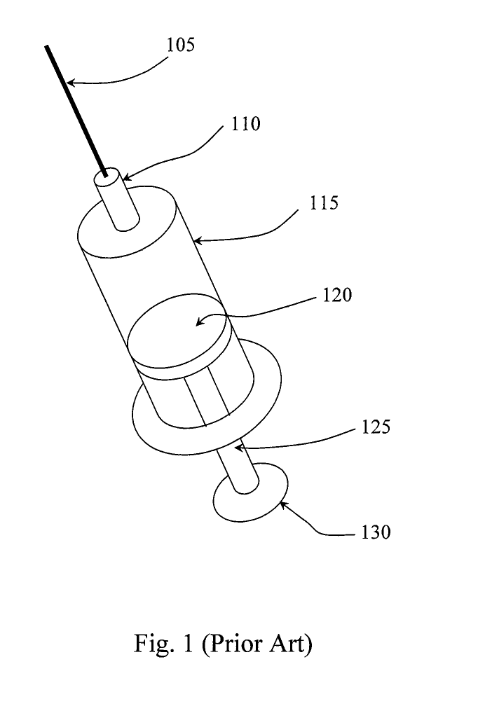

Controller 305 is typically an integrated circuit with power, input, and output pins capable of performing logic functions. In various embodiments, controller 305 is a targeted device controller. In such a case, controller 305 performs specific control functions targeted to a specific device or component, such as a heater or a power supply. For example, a heater controller has the basic functionality to control a heater. In other embodiments, controller 305 is a microprocessor. In such a case, controller 305 is programmable so that it can function to control more than one component of the device. In other cases, controller 305 is not a programmable microprocessor, but instead is a special purpose controller configured to control different components that perform different functions. In the embodiment of FIG. 3, controller 305 controls power supply 310 and reads data from memory device 315. While depicted as one component in FIG. 1, controller 305 may be made of many different components or integrated circuits.

FIG. 4 is an exploded cross section view of a drug delivery tip segment for an ophthalmic hand piece according to an embodiment of the present invention. In FIG. 4, the drug delivery tip segment includes housing 215, needle 210, optional light 275, fuse 375, plunger shaft 410, plunger tip 415, mechanical linkage interface 420, dispensing chamber 405, dispensing chamber housing 425, heater 450, thermal sensor 460, and optional luer 430.

In the embodiment of FIG. 4, mechanical linkage interface is located on one end of plunger shaft 410. Plunger tip 415 is located on the other end of plunger shaft 410. Plunger shaft 410 and plunger tip 415 collectively form a plunger. Dispensing chamber 405 is enclosed by dispensing chamber housing 425 and plunger tip 415. Plunger tip 415 forms a fluid seal with the interior surface of dispensing chamber housing 425. Needle 210 is fluidly coupled to dispensing chamber 405. In this manner, a substance located in dispensing chamber 405 can be contacted by plunger tip 415 and pushed out of needle 210. Needle 210 may be secured to the drug delivery tip segment by an optional luer 430 or may be permanently attached. Heater 450 is located on dispensing chamber housing 425 and at least partially surrounds dispensing chamber 405. Housing 215 forms an outer skin on the drug delivery tip segment and at least partially encloses plunger shaft 410, plunger tip 415, dispensing chamber 405, and dispensing chamber housing 425. Optional light 275 is visible from outside of housing 215. Optional light 275 may be illuminated, for example, when the tip segment is ready to be used. Fuse 375 is connected in series with optional light 275.

A substance to be delivered into an eye, typically a drug, is located in dispensing chamber 405. In this manner, the substance is contacted by the inner surface of dispensing chamber housing 425 and one face of plunger tip 415. Typically, dispensing chamber 405 is cylindrical in shape. Heater 450 is in thermal contact with dispensing chamber housing 425. In this manner, heater 450 is adapted to heat the contents of dispensing chamber 425. Current is applied to heater 450 through an electrical interface (not shown). Thermal sensor 460 provides temperature information to assist in controlling the operation of heater 450.

In one embodiment of the present invention, the substance located in dispensing chamber 405 is a drug that is preloaded into the dispensing chamber. In such a case, the drug delivery tip segment is appropriate as a single use consumable product. Such a disposable product can be assembled at a factory with a dosage of a drug installed. A precise volume of a substance can be preloaded into the delivery device.

When the drug is preloaded into dispensing chamber 405, a set quantity of the drug can be preloaded. For example, 100 microliters of a drug can be loaded into dispensing chamber 405, and any quantity up to 100 microliters can be dispensed. In such a case, the plunger (plunger shaft 410 and plunger tip 415) can be moved a precise distance to deliver a precise dosage of drug from the dispensing chamber 405, through the needle 210, and into an eye. This provides for flexibility of dosing and for ease of assembly.

In operation, the drug delivery tip segment of FIG. 4 is attached to a limited reuse assembly (not shown). The limited reuse assembly provides power to the tip segment and illuminates optional light 275. In such a case, a current passes through optional light 275 and fuse 375. Mechanical interface 420 mates with a mechanical interface on the limited reuse assembly. When a force is applied to plunger shaft 410, plunger tip 415 is displaced. The displacement of plunger tip 415 in turn displaces the substance contained in dispensing chamber 405. The substance is pushed out of needle 210. After the dosage is delivered, the controller (not shown) directs an increased current to be sent through fuse 375 and optional light 275. This increased current burns out fuse 375 indicating that the tip segment has been used and is to be discarded. Any number of commonly known methods can be used to increase the current to blow fuse 375. Since the tip segment of the depicted embodiment is a single use tip segment, once fuse 375 is blown, the tip segment is no longer operable.

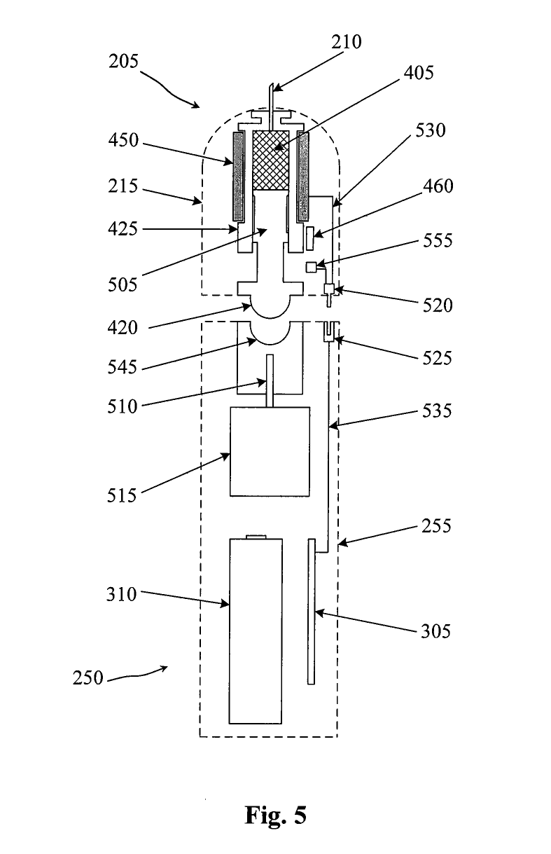

FIG. 5 is cross section view of a drug delivery tip segment and a limited reuse assembly according to an embodiment of the present invention. FIG. 5 shows how tip segment 205 interfaces with limited reuse assembly 250. In the embodiment of FIG. 5, tip segment 205 includes fuse assembly 555, mechanical linkage interface 420, plunger 505, dispensing chamber housing 425, tip segment housing 215, heater 450, thermal sensor 460, needle 210, dispensing chamber 405, interface 530, and tip interface connector 520. Limited reuse assembly 250 includes mechanical linkage 545, actuator shaft 510, actuator 515, power source 310, controller 305, limited reuse assembly housing 255, interface 535, and limited reuse assembly interface connector 525.

In tip segment 205, mechanical linkage 420 is located on one end of plunger 505. The other end of plunger 505 forms one end of dispensing chamber 405. Plunger 505 is adapted to slide within dispensing chamber 405. An outer surface of plunger 505 is fluidly sealed to the inner surface of dispensing chamber housing 425. Dispensing chamber housing 425 surrounds the dispensing chamber 405. Typically, dispensing chamber housing 425 has a cylindrical shape. As such, dispensing chamber 405 also has a cylindrical shape. In tip segment 205, fuse assembly 555 includes a fuse and an optional light connected in series as shown in FIG. 3.

Needle 210 is fluidly coupled to dispensing chamber 405. In such a case, a substance contained in dispensing chamber 405 can pass through needle 210 and into an eye. Heater 450 at least partially surrounds dispensing chamber housing 425. In this case, heater 450 is adapted to heat dispensing chamber housing 425 and any substance contained in dispensing chamber 405. In other words, heater 450 is in thermal contact with dispensing chamber housing 425. Interface 530 connects heater 450 with tip interface connector 520.

The components of tip segment 205, including dispensing chamber housing 425, heater 450, and plunger 505 are at least partially enclosed by tip segment housing 215. In one embodiment consistent with the principles of the present invention, a seal is present on a bottom surface of tip segment housing 215. In this manner, plunger 505 is sealed to tip segment housing 215. This seal prevents contamination of any substance contained in dispensing chamber 405. For medical purposes, such a seal is desirable. This seal can be located at any point on plunger 505 or on dispensing chamber housing 425. In such a case, tip segment housing 215 maybe connected to dispensing chamber housing 425 to form an air tight or fluid tight seal. In another embodiment, tip segment housing 215 may be sealed to plunger 505 near the end on which mechanical linkage interface 420 resides. In such a case, an air tight or fluid tight seal may be formed between a location on plunger 505 and tip segment housing 215.

In addition, tip segment 205 may contain a plunger stop mechanism. As shown in FIG. 5, the bottom portion of plunger 505 (the portion on which mechanical linkage interface 420 resides) is adapted to contact the bottom portion of dispensing chamber housing 425. In such a case, as plunger 505 advances upward toward needle 210, mechanical linkage interface 420 also advances upward toward needle 210. A top surface of mechanical linkage interface 420 contacts a bottom surface of dispensing chamber housing 425. In this embodiment, the protrusions on the bottom end on plunger 505 and the bottom surface of dispensing chamber housing 425 form a plunger stop mechanism. Plunger 505 cannot be advanced any further than the point at which the top surface of mechanical linkage interface 420 contacts the bottom surface of dispensing chamber housing 505. Such a plunger stop mechanism can provide a safety feature, such as to prevent plunger 505 from contacting needle 210 and possibly dislodging it. In another embodiment consistent with the principles of the present invention, such a plunger stop mechanism may also include a locking mechanism so that plunger 505 cannot be retracted or moved away from needle 210 when needle 210 is removed from the eye. Such a plunger lock mechanism helps to prevent reflux of the substance when needle 210 is removed.

In limited reuse assembly 250, power source 310 provides power to actuator 515. An interface (not shown) between power source 310 and actuator 515 serves as a conduit for providing power to actuator 515. Actuator 515 is connected to actuator shaft 510. When actuator 515 is a stepper motor, actuator shaft 510 is integral with actuator 515. Mechanical linkage interface 545 is connected to actuator shaft 510. In this configuration, as actuator 515 moves actuator shaft 510 upward toward needle 210 mechanical linkage 545 also moves upward toward needle 210.

Controller 305 is connected via interface 535 to limited reuse assembly interface connecter 525. Limited reuse assembly interface connecter 525 is located on a top surface of limited reuse assembly housing 255 adjacent to mechanical linkage interface 545. In this manner, both limited reuse assembly interface connector 525 and mechanical linkage interface 545 are adapted to be connected with tip interface connector 520 and mechanical linkage interface 420 respectively.

Controller 305 and actuator 515 are connected by an interface (not shown). This interface (not shown) allows controller 305 to control the operation of actuator 515. In addition, an optional interface (not shown) between power source 310 and controller 305 allows controller 305 to control operation of power source of 505. In such a case, controller 305 may control the charging and the discharging of power source 310 when power source 310 is a rechargeable battery. Controller 305 may also control the current provided to fuse assembly 555 in order to illuminate the optional light and blow the fuse. Controller 305 may also detect if the fuse has been blown.

Tip segment 205 is adapted to mate with or attach to limited reuse assembly 250 as previously described. In the embodiment of FIG. 5, mechanical linkage interface 420 located on a bottom surface of plunger 505 is adapted to connect with mechanical linkage interface 545 located near a top surface of limited reuse assembly housing 255. In addition, tip interface connector 520 is adapted to connect with limited reuse assembly interface connector 525. When tip segment 205 is connected to limited reuse assembly 250 in this manner, actuator 515 and actuator shaft 510 are adapted to drive plunger 505 upward toward needle 210. In addition, an interface is formed between controller 305 and heater 450. A signal can pass from controller 305 to heater 450 through interface 535, limited reuse assembly interface connector 525, tip interface connector 520, and heater interface 530.

In operation, when tip segment 205 is connected to limited reuse assembly 250, controller 305 controls the operation of actuator 515. Actuator 515 is actuated and actuator shaft 510 is moved upward toward needle 210. In turn, mechanical linkage interface 545, which is connected to mechanical linkage interface 420, moves plunger 505 upward toward needle 210. A substance located in dispensing chamber 405 is then expelled through needle 210.

In addition, controller 305 controls the operation of heater 450. Heater 450 is adapted to heat an outside surface of dispensing chamber housing 425. Since dispensing chamber housing 425 is at least partially thermally conductive, heating dispensing chamber housing 425 heats a substance located in dispensing chamber 405. Temperature information can be transferred from thermal sensor 460 through interface 530, tip interface connector 520, limited reuse assembly interface connector 525, and interface 535 back to controller 305. This temperature information can be used to control the operation of heater 450. Typically, controller 305 controls the amount of current that is sent to heater 450. The more current sent to heater 450, the hotter it gets. In such a manner, controller 305 can use a feed back loop utilizing information from thermal sensor 460 to control the operation of heater 450. Any suitable type of control algorithm, such as a proportional integral derivative (PID) algorithm, can be used to control the operation of heater 450.

Controller 305 is also adapted to operate fuse assembly 555. In this manner, controller 305 directs current to flow from power source 310 to fuse assembly 555. As previously depicted in FIG. 3, fuse assembly 555 includes a fuse and an optional light connected in series. A current passing through optional light 275 and fuse 375 illuminates optional light 275. After the tip segment 205 has been used (after the substance has been dispensed), controller 305 directs an increased current to blow fuse 375 and extinguish optional light 275. This indicates that the tip segment 205 has been used and that it should be discarded. In addition, controller 305 may check fuse 375 to see if it is blown. In such a case, tip segment 205 is rendered inoperable. Alternatively, fuse 375 may be placed such that when it is blown, no power is delivered to the tip segment. In such a case, once fuse 375 is blown, optional light 275 is extinguished and the tip segment is rendered inoperable. Other indicators on the limited reuse assembly 250 or the charging base (not shown) may indicate that the fuse 375 is blown.

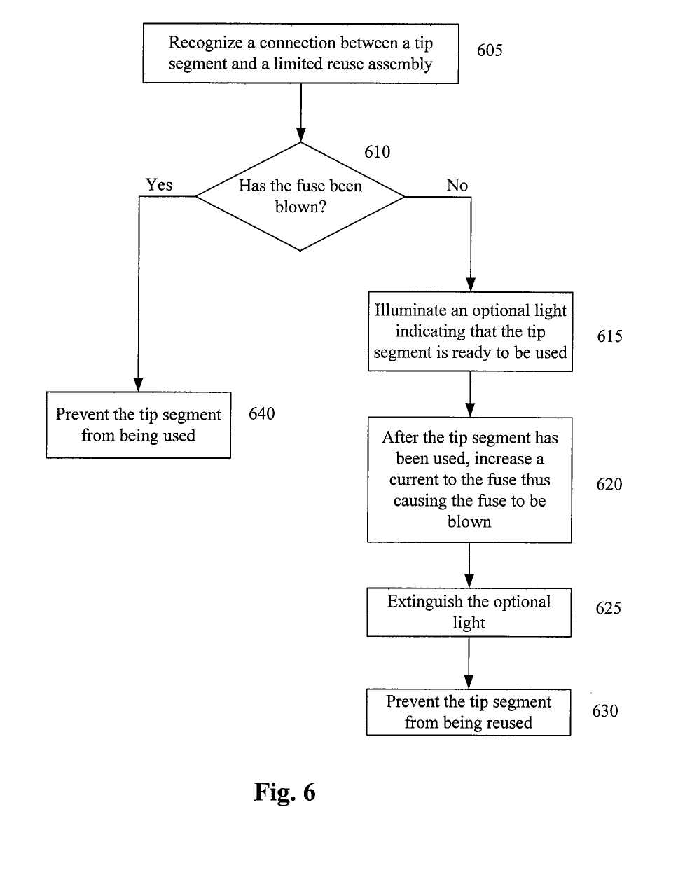

FIG. 6 is a flow chart depicting one method of operating the present invention. In 605 a connection between a tip segment and a limited reuse assembly is recognized. In 610, a determination is made as to whether or not a fuse has been blown. If the fuse has been blown, then in 640, the tip segment is prevented from being used. If the fuse has not been blown, then in 615, an optional light is illuminated indicating that the tip segment is ready to be used. In 620, after the tip segment has been used, the fuse is blown by an increased current. In 625, the optional light is extinguished. In 630, the tip segment is prevented from being reused.

From the above, it may be appreciated that the present invention provides an improved system and methods for delivering precise volumes of a substance into an eye. The present invention provides a single use, disposable delivery device tip segment that is capable of delivering a precise dosage. The tip segment interfaces with a universal hand piece limited reuse assembly. The disposable tip segment is provided with a fuse that indicates whether or not it is ready to be used. The fuse prevents the reuse of the disposable tip segment. The present invention is illustrated herein by example, and various modifications may be made by a person of ordinary skill in the art.

While the present invention is described in the context of a single-use drug delivery device, the present invention encompasses any single-use medical device that interfaces with a source of electric power. Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

What is claimed is:

1. A disposable injection device comprising:
a dispensing chamber having an inner surface and an outer surface, the inner surface defining a cavity for receiving a quantity of a substance;
a plunger engaged with the inner surface of the dispensing chamber, the plunger capable of sliding in the cavity of the dispensing chamber, the plunger fluidly sealed to the inner surface of the dispensing chamber;
a fuse; and
a housing at least partially enclosing the dispensing chamber and the plunger;
wherein after the substance has been delivered from the dispensing chamber, the fuse is blown disabling the device.

2. The device of claim 1 further comprising:
a light in series with the fuse.

3. The device of claim 1 further comprising:
a needle fluidly coupled to the dispensing chamber.

4. The device of claim 1 further comprising:
a heater in thermal contact with the dispensing chamber, the heater for heating the substance.

5. The device of claim 1 wherein the substance is a drug for treating a condition of the eye.

6. The device of claim 2 wherein the light is a light emitting diode.

7. The device of claim 1 further comprising:
a controller adapted to detect when the substance has been delivered from the dispensing chamber and direct the fuse to be blown.

8. The device of claim 2 further comprising:
a power source for providing a current to the light and the fuse.

9. The device of claim 9 wherein the power source is a battery.

10. The device of claim 1 further comprising:
a actuator for driving the plunger.

11. An ophthalmic injection system comprising:
a tip segment and a limited reuse assembly;
the tip segment comprising:
a dispensing chamber having an inner surface and an outer surface, the inner surface defining a cavity for receiving a quantity of a substance;
a plunger engaged with the inner surface of the dispensing chamber, the plunger capable of sliding in the cavity of the dispensing chamber, the plunger fluidly sealed to the inner surface of the dispensing chamber, the plunger having a proximate end and a distal end; the proximate end having a first mechanical linkage interface;
a fuse; and
a first housing at least partially enclosing the dispensing chamber and the plunger;
the limited reuse assembly comprising:
a power source;
a controller for controlling the operation of the system;
a actuator having a shaft;
a second mechanical linkage interface located on an end of the shaft; and
a second housing at least partially enclosing the controller and the actuator;
wherein after the substance has been delivered from the dispensing chamber, the fuse is blown disabling the device.

12. The device of claim 11 further comprising:
a light connected in series with the fuse.

13. The system of claim 11 further comprising:
an electrical interface for coupling the heater to the limited reuse assembly.

14. The system of claim 11 wherein the controller operates the actuator such that the shaft is moved a calculated distance thereby displacing the plunger and causing a fixed amount of the substance to exit the dispensing chamber.

15. The device of claim 11 further comprising:
a needle fluidly coupled to the dispensing chamber.

16. The device of claim 11 further comprising:
a heater in thermal contact with the dispensing chamber, the heater for heating the substance.

17. The device of claim 11 wherein the substance is a drug for treating a condition of the eye.

18. The device of claim 12 wherein the light is a light emitting diode.

19. The device of claim 11 wherein the controller is adapted to detect when the substance has been delivered from the dispensing chamber and direct the fuse to be blown.

20. The device of claim 11 wherein the power source is a battery.

21. A method of operating an ophthalmic injection system comprising:
recognizing a connection between a tip segment and a limited reuse assembly;
determining if a fuse has been blown;
if the fuse has not been blown, indicating that the tip segment is ready to be used; and
after the tip segment has been used, increasing a current to the fuse thus causing the fuse to be blown.

22. The method of claim 21 further comprising:
extinguishing a light after the tip segment has been used.

23. The method of claim 21 further comprising:
detecting that the fuse has been blown; and
preventing the tip segment from being used.

24. A medical device comprising:
a single-use component comprising a fuse connected in series with a light and an interface for receiving electric current, the electric current being provided to the fuse and the light; and
a limited reuse assembly attachable to and removable from the single-use component, the limited reuse assembly comprising a controller for controlling the operation of the single-use component and a power source for providing the electric current to the fuse and the light via the interface;
wherein after the single-use component has been used, the fuse is blown thus disabling the single use component.

25. The device of claim 24 wherein the single use component further comprises:
a dispensing chamber having an inner surface and an outer surface, the inner surface defining a cavity for receiving a quantity of a substance;
a plunger engaged with the inner surface of the dispensing chamber, the plunger capable of sliding in the cavity of the dispensing chamber, the plunger fluidly sealed to the inner surface of the dispensing chamber;

26. A single-use medical device comprising:
a fuse connected in series with a light;
an interface for receiving electric current, the electric current being provided to the fuse and the light;
wherein after the device has been used, the fuse is blown thus disabling the device.
